(19) United States
(12) Patent Application Publication
(10) Pub. No.: US 2023/0063220 A1
(43) Pub. Date: Mar. 2, 2023

(54) Systems and Methods for Predicting Missing Data Values

(71) Applicant: EnableComp, LLC, Franklin, TN (US)

(72) Inventors: Kevin Brent Hulvey, Winchester, TN (US); Alan Cunningham, Murfreesboro, TN (US); Justin Crockett, Ashland City, TN (US); Craig Connally, Franklin, TN (US); Michael Bies, Franklin, TN (US)

(21) Appl. No.: 17/446,415

(22) Filed: Aug. 30, 2021

(51) Int. Cl. G06Q 40/08; G16H 40/20; G06Q 50/26; G06Q 10/10; G16H 10/60; G06F 16/23; G06F 16/9538

(52) U.S. Cl. CPC G06Q 40/08; G16H 40/20; G06Q 50/265; G06Q 10/1057; G16H 10/60; G06F 16/2365; G06F 16/9538

(57) ABSTRACT

A method may include receiving a file with a first healthcare claim. The method may include parsing the first healthcare claim into one or more claim data elements. The method may include performing an automatic discovery process on a first claim data element of the one or more data elements. The automatic discovery process may include identifying a replacement value for the claim data element value of the first claim data element and assigning an accuracy score to the replacement value. In response to the accuracy score exceeding a pre-determined score threshold, the automatic discovery process may include replacing the claim data element value of the first claim data element with the replacement value. In response to the accuracy probability score being below the pre-determined score threshold, the automatic discovery process may include presenting the replacement value via a user interface.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND OF THE DISCLOSURE

Workers' compensation healthcare claims and Veterans Health Administration (VA) healthcare claims are much more complex than typical health insurance healthcare claims. For example, unlike typical health insurance claims, there is no national database for workers' compensation coverage eligibility. Additionally, when seeking healthcare treatment, most employees do not know who their employer's workers' compensation company is. These companies are usually only known by the employer's human resources or financial departments. Healthcare providers often do not understand what payment rules are in place for these claims due to the highly complex nature of state-specific workers' compensation fee schedules and tables. VA eligibility and processing rules are also complex in nature and often require very specific knowledge of regional processing rules in addition to prior authorization processes that must be followed. Because of these numerous technical shortfalls and challenges, many workers' compensation and VA healthcare claims are created incorrectly and, thus, are denied or paid less than legally directed.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is a computer-implemented method. The method may include receiving a file. The file may include a first healthcare claim. The method may include parsing the first healthcare claim into one or more claim data elements. Each claim data element may include a claim data element field and a claim data element value that corresponds to the claim data element field. The method may include performing an automatic discovery process on a first claim data element of the one or more data elements. The automatic discovery process may include identifying a replacement value for the claim data element value of the first claim data element. The automatic discovery process may include assigning an accuracy score to the replacement value. In response to the accuracy score exceeding a pre-determined score threshold, the automatic discovery process may include replacing the claim data element value of the first claim data element with the replacement value. In response to the accuracy probability score being below the pre-determined score threshold, the automatic discovery process may include presenting the replacement value via a user interface.

Numerous other objects, advantages and features of the present disclosure will be readily apparent to those of skill in the art upon a review of the following drawings and description of various embodiments.

### BRIEF DESCRIPTION OF THE DRAWINGS

FIG. 1 is a schematic block diagram illustrating one embodiment of a system for predicting missing data values.

FIG. 2 is a schematic block diagram illustrating one embodiment of a server for a system for predicting missing data values.

FIG. 3 is a flowchart diagram illustrating one embodiment of a method for predicting missing data values.

FIG. 4 is a flowchart diagram illustrating one embodiment of an automatic discovery process that is part of a method for predicting missing data values.

FIG. 5 is a schematic block diagram illustrating one embodiment of a healthcare claim.

FIG. 6 is a schematic block diagram illustrating one embodiment of a user interface for use in systems and methods for predicting missing data values.

### DETAILED DESCRIPTION

While the making and using of various embodiments of the present disclosure are discussed in detail below, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatus and methods described herein. Such equivalents are considered to be within the scope of this disclosure and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Reference throughout this specification to “one embodiment,” “an embodiment,” “another embodiment,” or similar language means that a particular feature, structure, or characteristic described in connection with the embodiment is included in at least one embodiment. Thus, appearances of the phrases “in one embodiment,” “in an embodiment,” “in some embodiments,” and similar language throughout this specification may, but do not necessarily, all refer to the same embodiment, but mean “one or more but not necessarily all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. As used herein, the term “a,” “an,” or “the” means “one or more” unless otherwise specified. The term “or” means “and/or” unless otherwise specified.

Multiple elements of the same or a similar type may be referred to as “Elements 102(1)-(n)” where n may include a number. Referring to one of the elements as “Element 102” refers to any single element of the Elements 102(1)-(n). Additionally, referring to different elements “First Elements 102(1)-(n)” and “Second Elements 104(1)-(n)” does not necessarily mean that there must be the same number of First Elements as Second Elements and is equivalent to “First Elements 102(1)-(n)” and “Second Elements (1)-(m)” where m is a number that may be the same or may be a different number than n.

As used herein, the term “computing device” includes a device configured to receive data, process the data, and output a result based on that processing. A computing device may include technological components such as a processor, a memory, a data storage, an input device, and output device, a communications interface, or other components included in computing devices. A computing device may include a desktop computer, a laptop computer, a mobile device (e.g., a mobile phone, a tablet computer, a smart watch, etc.), or a workstation. A computing device may include an application server, a database server, or some other type of server. In one or more embodiments, a computing device may include a virtual machine (VM) that is executable on a physical computing device. In one embodiment, a computing device may include a system of multiple computing devices networked together. Such a computing device may include a distributed computing system, a cloud computing system, a supercomputer, or some other system.

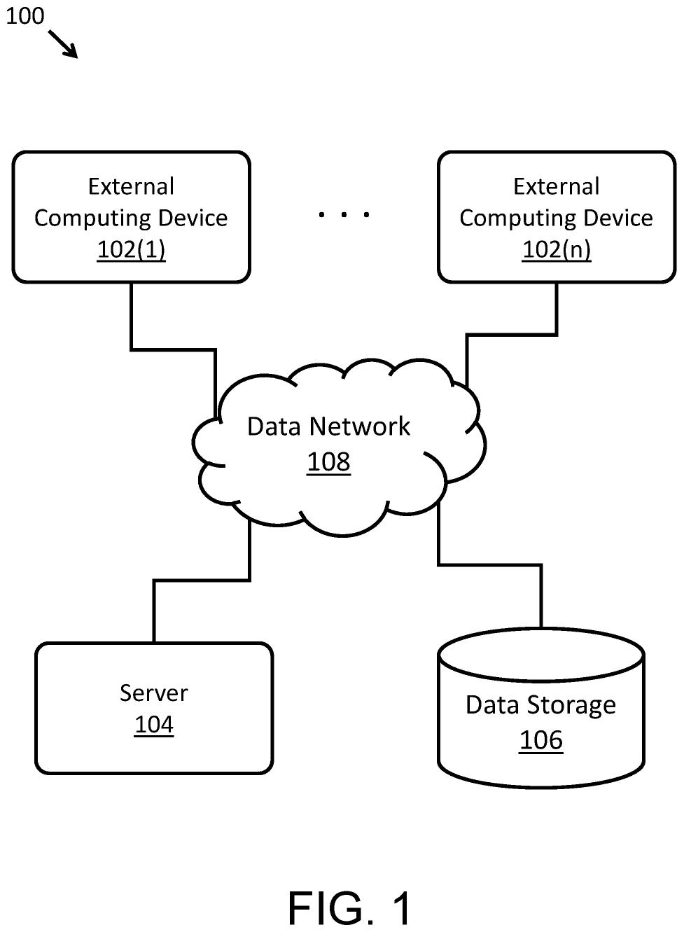

FIG. 1 depicts one embodiment of a system 100. The system 100 may include a system for predicting missing data values. The system 100 may include one or more external computing devices 102(1)-(n). The system 100 may include a server 104. The system 100 may include a data storage 106. The system 100 may include a data network 108. Further details regarding the system 100 and its components are discussed below.

In some embodiments, the external computing device 102(1) may include a computing device. The external computing device 102(1) may include a computing device at a medical facility. The medical facility may include a hospital, a doctor's office, an outpatient clinic, a long-term care facility, a clinical lab, a hospice facility, an ambulatory surgical center, a dialysis facility, or some other type of medical facility. At the medical facility, a worker may input information into the external computing device 102(1) to generate a healthcare claims file. In some embodiments, the external computing device 102(1) may include a computing device located at a healthcare clearinghouse. The healthcare clearinghouse may receive data from a medical facility and generate a healthcare claims file based on the received data. In some embodiments, the external computing device 102(1) may be “external” in the sense that the external computing device 102(1) is external from the server 104 or the data storage 106. The external computing device 102(1) being external from the server 104 or the data storage 106 may include the external computing device 102(1) being logically external, physically external, or controlled by a different entity than the server 104 or the data storage 106.

In some embodiments, the server 104 may include a computing device. The server 104 may receive a healthcare claims file from the external computing device 102(1). The server 104 may process the healthcare claims file. Processing the healthcare claims file may include various processes such as correcting errors in the file, matching claims with previous healthcare encounters of a similar type, determining whether to augment the claims with data from the previous healthcare encounters, and storing data during the processing to aid with the processing. The server 104 may generate a separate healthcare claims file that may include one or more augmented healthcare claims. The server 104 may send this healthcare claims file to a clearinghouse or a healthcare insurance provider.

In some embodiments, the data storage 106 may include a database, a file system, a data lake, a data warehouse, cloud storage, or some other type of data storage. The server 104 may include the data storage 106, or the data storage 106 may be physically or logically separate from the server 104. The data storage 106 may store various types of data. For example, the data storage 106 may store the healthcare claims file received from the external computing device 102(1), healthcare claims data based on the healthcare claims file, augmented healthcare claims data, or other types of data.

In one embodiment, the data network 108 may include a wired network or a wireless network. The data network 108 may include one or more routers, switches, gateways, or other network devices. The data network 108 may include one or more local area networks (LANs), wide area networks (WANs), or other networks. The data network 108 may include an Internet service provider (ISP), the Internet, or some other network. The data network 108 may facilitate digital data exchange or communication between the one or more external computing devices 102(1)-(n), the server 104, or the data storage 106.

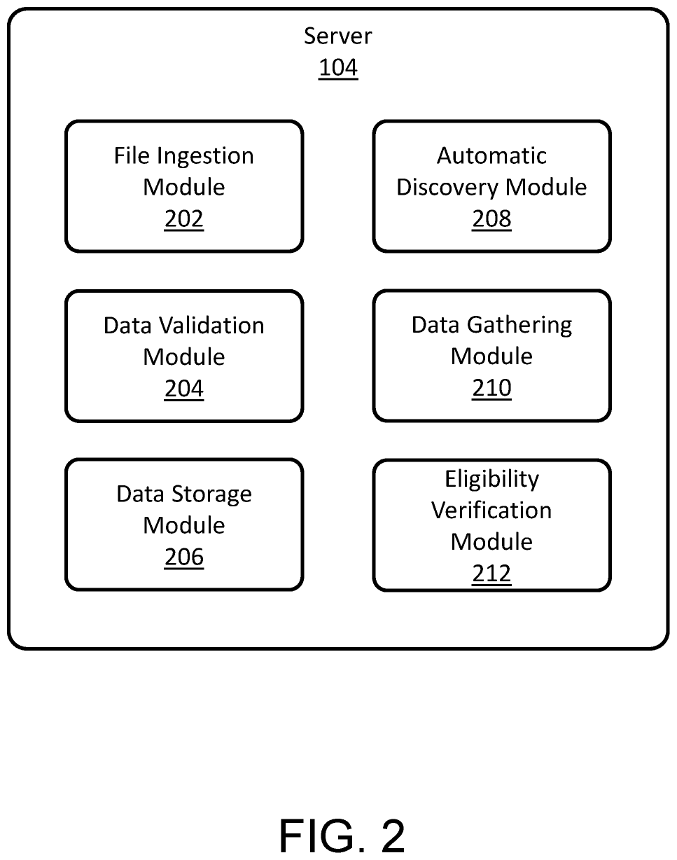

FIG. 2 depicts one embodiment of the server 104. The server 104 may include a file ingestion module 202. The server 104 may include a data validation module 204. The server 104 may include a data storage module 206. The server 104 may include an automatic discovery module 208. The server 104 may include a data gathering module 210. The server 104 may include an eligibility verification module 212.

In some embodiments, the file ingestion module 202 may import a file into the server 104. The file may include a healthcare claims file or some other type of file. The file ingestions module 202 may generate data based on the imported file, such as claim data elements (discussed further below). In one embodiment, the data validation module 204 may validate data imported into the server, such as the imported file, the data generated based on the imported file, data imported via the data gathering module 210, or other data. In certain embodiments, the data storage module 206 may send data from the server 104 to the data storage 106 or may retrieve data from the data storage 106 and provide the retrieved data to the server 104. The data storage module 206 may include a database management system (DBMS) or some other type of data storage software.

In one embodiment, the automatic discovery module 208 may perform an automatic discovery process (discussed further below) to augment or supplement the data imported into the server 104. The data gathering module 210 may retrieve data from one or more external computing devices 102(1)-(n) in order to store the retrieved data in the data storage 106 or aid the automatic discovery module 208. In some embodiments, the eligibility verification module 212 may provide patient eligibility verification functionality to one or more external computing devices 102(1)-(n). The eligibility verification module 212 may include an application programming interface (API) via which the one or more external computing devices 102(1)-(n) may interact with the eligibility verification module 212. Further details about the functionality of the modules 202-212 of the server 104 are discussed further below.

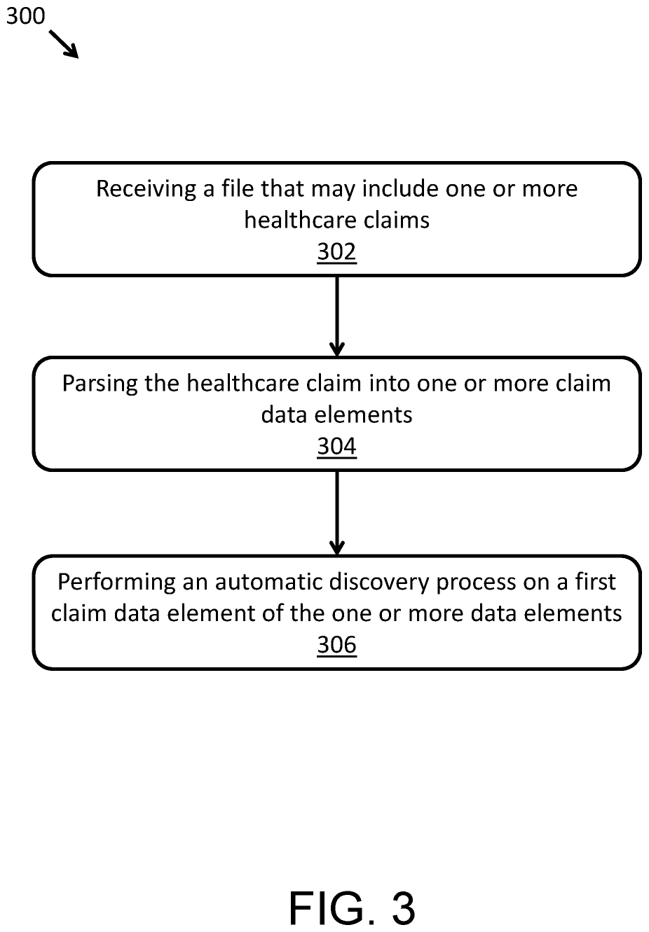

FIG. 3 depicts one embodiment of a method 300. The method 300 may include a method for predicting missing data values. The method 300 may be performed by one or more components of the system 100 of FIG. 1 or one or more modules 202-212 of the server 104. The method 300 may include receiving 302 a file. The file may include a first healthcare claim. The method 300 may include parsing 304 the first healthcare claim into one or more claim data elements. Each claim data element may include a claim data element field. Each claim data element may include a claim data element value. The claim data element value may correspond to the claim data element field. The method 300 may include performing 306 an automatic discovery process on a first claim data element of the one or more data elements.

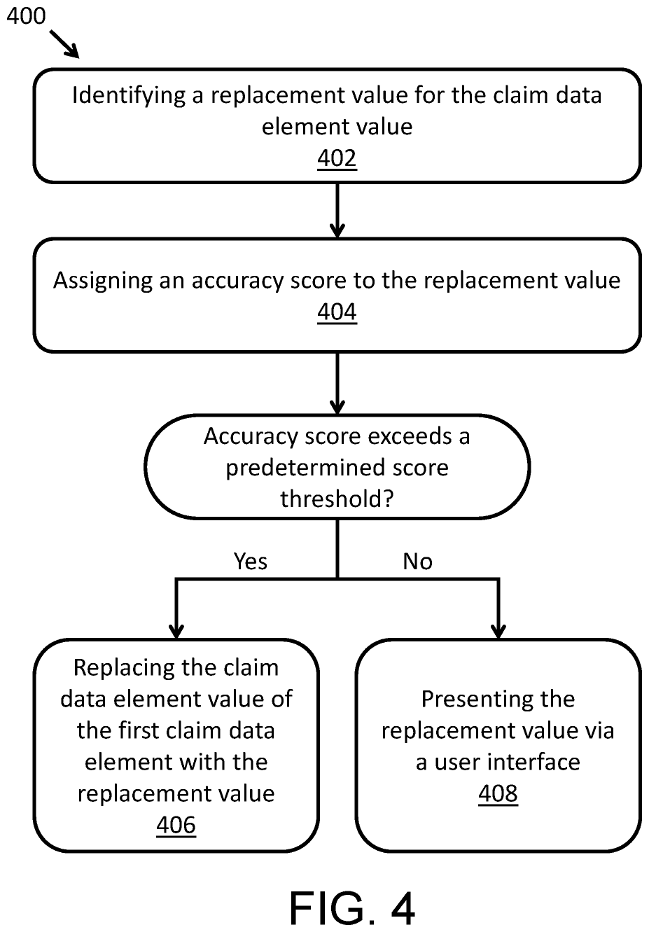

FIG. 4 depicts one embodiment of a method 400. The method 400 may include a method for the automatic discovery process of the step 306 of the method 300 of FIG. 3. In one embodiment, the method 400 may include identifying 402 a replacement value for the claim data element value of the first claim data element. The method 400 may include assigning 404 an accuracy score to the replacement value. In some embodiments, the method 400 may include, in response to the accuracy score exceeding a pre-determined score threshold, replacing 406 the claim data element value of the first claim data element with the replacement value. The method 400 may include, in response to the accuracy probability score being below the pre-determined score threshold, presenting 408 the replacement value via a user interface.

Returning to FIG. 3, in some embodiments, the method 300 may include receiving 302 a file. In one embodiment, the file may include an X12 file, an ANSI 835 file, an ANSI 837 file, a flat file, a Uniform Billing Form (UB-04) file, a Health Care Financial Administration (HCFA) file, or some other type of file that may include data embodying the first healthcare claim. The file may include the first healthcare claim. A healthcare claim may include information or data regarding a request for payment that a patient or healthcare provider may submit to a payer for direct payment or reimbursement for medical services that the healthcare provider rendered to the patient. In one or more embodiments, a healthcare claim may include a workers' compensation claim, a VA healthcare claim, a health insurance claim, a medical claim, a dental claim, or some other type of claim.

In one or more embodiments, receiving 302 the file may include the server 104 receiving the file over the data network 108 from the external computing device 102(1). Receiving the file over the data network 108 may include using a secure file transfer protocol (secure FTP) to receive the file. In some embodiments, the file ingestion module 202 may receive the file. Receiving 302 the file may include the file ingestion module 202 sending the file to the data storage module 206 to store the file in the data storage 106. In one embodiment, receiving 302 the file may include the file ingestion module 202 translating the file into a different format. The format may include a different file format, a different healthcare claims format, or some other type of format.

In one embodiment, the method 300 may include parsing 304 the first healthcare claim into one or more claim data elements. In some embodiments, the file ingestion module 202 may perform the parsing 304. A claim data element may include a portion of a healthcare claim. A portion of a healthcare claim may include a subset of the data contained within the healthcare claim.

Examples of a claim data element may include a patient name, an employer name, a patient Social Security number, a payer name, a payer address, a payer identifier, a billing provider name, a billing provider address, a billing provider identifier, a healthcare provider name, a healthcare provider address, a national provider identifier (NPI), a tax ID, a service location, a claim number, an injury date, a service date, a billing location identifier, a subscriber relation code, a claim creator, a claim receiver, a rendering provider, a referring provider, an information contact, a claim casualty number, a standard current procedural terminology (CPT) code, a principle diagnosis, a diagnosis, or other claim data elements.

In some embodiments, a claim data element may include a claim data element field. The claim data element field may include data identifying the type of claim element. For example, a claim data element field may include “Patient Name.” A claim data element may include a claim data element value. The claim data element value may correspond to the claim data element field. The claim data element value may include the actual value for the field. As an example, the claim data element value corresponding to the claim data element field “Patient Name” may include “John Albert Smith.” In some embodiments, the claim data element including the claim data element field may include the claim data element including data indicating or referencing the claim data element field without including the claim data element field directly.

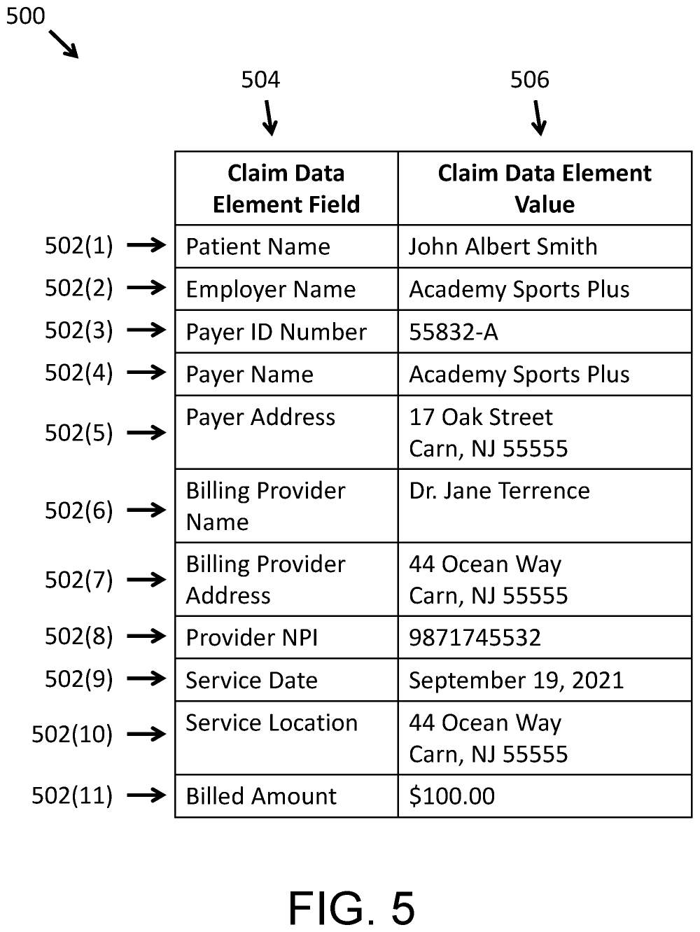

FIG. 5 depicts one example embodiment of the first healthcare claim 500. As can be seen from FIG. 5, the first healthcare claim 500 may include one or more claim data elements 502(1)-(11). Each claim data element 502 may include a claim data element field 504 and a corresponding claim data element value 506. For example, claim data element 502(1) may include the field 504 “Patient Name,” and the corresponding value 506 may include “John Albert Smith.” This claim data element 502(1) may include the name of the patient for whom medical services were rendered. Claim data element 502(2) may include the field 504 “Employer Name,” and the corresponding value 506 may include “Academy Sports Plus.” This claim element 502(2) may include the name of the patient's employer. The claim data element 502(3) may include the field 504 “Payer ID Number,” and the corresponding value 506 may include “55832-A.” This claim data element 502(3) may include the ID number associated with the payer that uniquely identifies the patient from among all subscribers of the payer. The claim data element 502(4) may include the field 504 “Payer Name,” and the corresponding value 506 may include “Academy Sports Plus.” This claim data element 502(4) may include the name of the payer. The claim data element 502(5) may include the field 504 “Payer Address,” and the corresponding value 506 may include “17 Oak Street, Cam, N.J. 55555.” This claim data element 502(5) may include the address of the payer. In this example healthcare claim 500, the values 506 for the claim data elements 502(4)-(5) (“Payer Name” and “Payer Address”) may be incorrect due to, for example, the patient not being sure of who his workers' compensation insurance provider is (and, thus, putting down his employer) or due to the person inputting the healthcare claim information making a mistake and inputting the wrong information.

The claim data element 502(6) may include the field 504 “Billing Provider Name,” and the corresponding value 506 “Dr. Jane Terrence.” This claim data element 502(6) may include the name of the healthcare provider providing services to the patient (which may include an entity or a person). The claim data element 502(7) may include the field 504 “Billing Provider Address,” and the corresponding value 506 may include “44 Ocean Way, Cam, N.J. 55555.” This claim data element 502(7) may include the address of the billing healthcare provider. The claim data element 502(8) may include the field 504 “Provider NPI,” and the corresponding value 506 may include “9871745532.” This claim data element 502(8) may include the National Provider Identifier (NPI) of the provider that rendered services to the patient. In some embodiments, this claim data element 502(8) may include some other identifier that may assist in uniquely identifying the healthcare provider.

The claim data element 502(9) may include the field 504 “Service Date,” and the corresponding value 506 “Sep. 19, 2021.” This claim data element 502(9) may include the date on which the healthcare provider rendered healthcare services to the patient. The claim data element 502(10) may include the field 504 “Service Location,” and the corresponding value 506 “44 Ocean Way, Carn, N.J. 55555.” This claim data element 502(10) may include the address at which the healthcare provider rendered the healthcare services to the patient. The claim data element 502(11) may include the field 504 “Billed Amount,” and the corresponding value 506 “$100.00.” This claim data element 502(11) may include the amount the healthcare provider billed for rendering the healthcare services.

In some embodiments, a healthcare claim 500 may include more or fewer claim data elements 502 and may include different claim data elements 502 than those depicted in FIG. 5. In one embodiment, claim data elements 502 may include differing levels of granularity. For example, as depicted in FIG. 5, the claim data element 502(1) includes one claim data element 502 for the patient's name, but in some embodiments, the patient's name may be divided into multiple claim data elements 502 (e.g., one claim data element 502 for “First Name,” one for “Middle Name,” and one for “Last Name). In one embodiment, a healthcare claim 500 may include multiple instances of the same data element 502. For example, a healthcare claim 500 may include multiple service date claim data elements 502(9). This may indicate that treatment of the patient was spread over multiple days. In one or more embodiments, a claim data element 502 may include a different format, such as a range. For example, the service date claim data elements 502(9) may include a range of dates, which may indicate that treatment of the patient was spread over multiple days.

Returning to FIG. 3, in some embodiments, parsing 304 the first healthcare claim 500 into the one or more claim data elements 502 may include extracting the first healthcare claim 500 from the received file. Extracting the first healthcare claim 500 may include the file ingestion module 202 reading the file, identifying data that corresponds to a claim data element field 504, and generating a claim data element value 506 based on the identified data. In some embodiments, the file ingestion module 202 may include one or more data mapping rules. A mapping rule may include data indicating that a certain piece of identified data is mapped to a certain claim data element field 504. In one embodiment, the file ingestion module 202 may include client-specific data mapping rules. A client-specific mapping rule may transform data from a format in the received file to a standard, internal data layout. The file ingestion module 202 may include payer-specific mapping rules that may also transform data from a format in the received file to a standard, internal data layout. As an example, a client may store a data element in an X12 field according to its own format instead of what the payer may anticipate. In some embodiments, a received file may include data with limited accuracy or limited types of data. The file ingestion module 202 generating the claim data element value 506 may adjust the limited data into a different format or value.

In one embodiment, the method 300 may include storing the one or more claim data elements 502 in the data storage 106. The file ingestion module 202 may send the one or more claim data elements 502 to the data storage module 206 in order to store the one or more claim data elements in the data storage 106. In some embodiments, the data storage module 206 may store the one or more claims data elements 502 in a standardized data schema. The standardized data schema may be patterned after the ANSI X12 835. The standardized data schema may further be patterned on another schema that may house certain claim data elements 502. The standardized schema may provide for a more efficient sorting and processing of rules and reporting.

In one embodiment, the method 300 may include identifying a claim data element 502 that may include an error. Identifying an error in a claim data element 502 may occur prior to the claim data element 502 being stored in the data storage 106, after the claim data element 502 has been stored in the data storage 106, or at some other point in time. In some embodiments, the data validation module 204 may identify the error in the claim data element 502.

One type of error may include an absent claim data element 502 that is a required claim data element 502. A required claim data element 502 may include a claim data element 502 that must be present after the parsing 304 step.

Another type of error may include that a claim data element 502 includes data that is in an incorrect format. Data being in an incorrect format may include the claim data element 502 including alphabetic characters when numeric characters are expected. For example, the claim data element 502 for the field 504 “Social Security Number” may expect only numeric characters and may include an error if the corresponding value 506 includes alphabetic text such as “apple.” This may apply to claim data elements 502 for ZIP code, payer ID number, provider NPI, billed amount, or other typically numerical claim data elements 502. Data being in an incorrect format may include the claim data element value 506 including numeric characters when alphabetic characters are expected. For example, the claim data element 502(1) for the field 504 “Patient Name” may expect only alphabetic characters and may include an error if the corresponding value 506 includes numeric text such as “8751184.” This may apply to claim data elements 502 for employer name, payer name, billing provider name, or other typically alphabetic claim data elements 502.

Data being in an incorrect format may include the claim data element value 506 not being in an expected format. An expected format may include an address format (e.g., street number, street name, suite number, city, state, ZIP code), a name format (e.g., first name, middle name, last name), a business name (e.g., the name of business followed by “Company,” “Co.,” “Limited,” “LLC,” “Incorporated,” “Inc.,” etc.), a date format (month-day-year, year-month-day, etc.), or some other format. Data being in an incorrect format may include the claim data element value 506 including too many or too few characters. For example, the claim data element 502 for the filed 504 “Social Security Number” may include more than or fewer than nine characters.

Another type of error may include that the values 506 of multiple claim data elements 502 have been transposed. For example, the claim data element 502(1) for the field 504 “Patient Name” may include the value 506 “Academy Sports Plus,” and the claim data element 502(2) for the field 504 “Employer Name” may include the value 506 “John Albert Smith.”

In one embodiment, the data validation module 204 may include an error rules engine. The error rules engine may detect an error in a claim data element 502 or a healthcare claim 500 and, in response, perform functionality to address the detected error. The error rules engine may include one or more rules. Each rule may include a condition and a corresponding action.

In some embodiments, the condition may include detecting one or more of the errors discussed above. The data validation module 204 may detect the error via a regular expression, string-searching algorithm, or some other search pattern detection. The data validation module 204 determine the transposition of claim data elements 502 by comparing the values 506 against a set of known acceptable values such as a list of known payers, employers, or other entities. The data validation module 204 may detecting absent required claim data elements 502 by comparing the one or more claim data elements to a list of required filed 504.

In one or more embodiments, in response to identifying an error in a claim data element 502, the method 300 may include modifying the identified claim data element 502 to correct the error. The action of a rule may include functionality that modifies the identified claim data element 502 to correct the error. In response to the error including a missing claim data element 502, modifying the claim data element 502 may include generating the missing claim data element 502. The data validation module 204 may generate the missing claim data element 502 based on similar claim data elements 502 in the data storage 106. As an example, the data validation module 204 may identify that the first healthcare claims file is missing the patient's name. This may result in a missing patient name claim data element 502(1) for the healthcare claim 500. However, the file ingestion module 202 may have also parsed 304 a Social Security number claim data element 502 included in the first healthcare claims file. The data storage 106 may include a Social Security data element 502 with the same value 506 (because, for example, the server 104 may have received a second healthcare claims file associated with the same patient in the past). The Social Security data element 502 parsed 304 from the second healthcare claims file may be associated with a patient name data element 502(1) that was also parsed 304 from the second healthcare claims file. As a result, the data validation module 204 may use the patient name data element 502(1) from the second healthcare claims file to generate a patient name claim data element 502(1) for the first healthcare claims file since the patient names should be the same since the Social Security numbers are the same. A similar process can be used to correct other missing claim data elements 502 (e.g., correcting a missing provider name based on an NPI).

In some embodiments, in response to the error including a claim data element 502 including data in the wrong format, modifying the claim data element 502 may include modifying the claim data element 502 based on similar claim data elements 502 in the data storage 106. This may be similar to adding a missing claim data element 502, as discussed above. In some embodiments, in response to the error including a claim data element 502 including data in the wrong format, modifying the claim data element 502 may include determining if the claim data element 502 was entered into the wrong portion of the healthcare claims file. For example, after parsing 304 the healthcare claims file, the data validation module 204 may determine that the patient name claim data element 502(1) includes the value 506 “100 Oak Street” and that the patient's street address claim data element 502 includes the value 506 “John Albert Smith.” Using regular expressions, a rules engine, or other functionality, the data validation module 204 may determine that “100 Oak Street” is more likely to be a street address and “John Albert Smith” is more likely to be a name. The data validation module 204 may then determine, based on this information, that these values 506 were likely accidently transposed in the healthcare claims file and may correct the errors by exchanging their claim data element values 506.

In some embodiments, a rule may apply to all claim data elements 502 generated by the server 104. In other embodiments, a rule may only apply to claim data elements 502 generated from a file received from a specific entity. In certain embodiments, a rule may only apply to claim data elements 502 for a healthcare claim 500 that is of a certain class (e.g., workers' compensation, VA, etc.).

In one embodiment, the method 300 may include performing 306 the automatic discovery process on a first claim data element 502 of the one or more data elements 502(1)-(n). As discussed above, the automatic discovery process of the step 306 may include the method 400 of FIG. 4. The automatic discovery module 208 may perform one or more steps of the method 400. In one embodiment, the method 400 may include identifying discovery data. Discovery data may include data that may include one or more replacement values 506 that can be used in the automatic discovery process.

In some embodiments, the discovery data may include a second healthcare claim 500. In some embodiments, the second healthcare claim 500 may include a healthcare claim 500 that has already been processed by the server 104, that may already be stored in the data storage 106 (or at least a portion of its claim data elements 502 are included in the data storage 106), or may include another type of healthcare claim 500.

In certain embodiments, the discovery data may include data gathered by the data gathering module 210 of the server 104. The data gathering module 210 may gather the data from an external computing device 102(2), which may include an external computing device 102 different from the external computing device 102(1) that sent the file in step 302 of the method 300.

In one embodiment, the data gathering module 210 may execute a robot process automation (RPA) process to gather the data. The RPA process may access a website of an entity, such as a payer, a government organization, an employer, or some other entity. The RPA process may send virtualized key strokes, mouseclicks, touchscreen taps, or other virtualized inputs to a login screen of the website in order to login to the website. The RPA process may send inputs to the website to navigate through the website screens. The RPA process may send inputs to enter search data and virtually click buttons to enact searches and navigation. The website may present a search screen to the RPA process, and the search screen may include search results. The data gathering module 210 may extract, parse, and interpret data received from the website. The RPA process may send the received data to the data storage module 206 to be stored as discovery data. In some embodiments, the data gathering module 210 may retrieve one or more claims 500 in the data storage 106 that may match the received data. In other embodiments, the data gathering module 210 may send the received data to the data validation module 204 or the data storage module 206 to generate or store a claim 500 based on the received data. The data gathering module 210 may apply one or more claim processing rules to the claim 500 (whether retrieved from the data storage 106 or newly generated), and the application of the rule may cause the server 104 to process the claim 500. For example, the data gathering module 210 may receive payment information (e.g., payment amount, check number, check date, or other payment information) for a claim from a website. The data gathering module 210 may retrieve the claim 500 from the data storage 106 or may cause the server 104 to generate a new claim 500 based on the payment information. The server 104 may validate that the payment has been received by the hospital associated with the claim 500 and that the payment amount matched the expected amount. In response to the validation determining that the payment was denied or was below the expected amount, the server 104 may move the claim 500 into a denial/appeal work queue. Although the RPA process has been discussed in relation to a website, the same functionality is applicable to a web portal, a software application, a mobile application, or some other application capable of data communication with a payer's, a government organization's, an employer's, or another entity's system.

In some embodiments, the data gathering module may receive data from the external computing device 102(2) using an API of the external computing device 102(2). The external computing device 102(2) may include a database provided by a payer, a government organization, an employer, or some other entity. The external computing device 102(2) may include an API via which the data gathering module 210 may query the database and receive data in return. The data gathering module 210 may send the gathered data to the data storage module 206 to be stored as discovery data.

In some embodiments, the discovery data may include employer data. Employer data may include an employer name, an employer address, or data related to an entity that an employer has contracted with to provide workers' compensation insurance (such as the entity's name, address, subscriber IDs and their associated employee information). Employer data may include a set of employees of the employer, and the employee data may include the employees' respective names, addresses, contact information, subscriber IDs, Social Security numbers, or other employee data.

In some embodiments, the discovery data may include payment information. Payment information may include a payment amount, a payment line item that may match a service code on a bill, a payment date, or a check number. In one embodiment, payment information may include an adjustor name or adjustor contact information. In one or more embodiments, payment information may include a payment amount of zero and may further include some indication of a denial or denial reasons. In some embodiments, discovery data may include an authorization number. An authorization number may include a number that may indicate that the medical service has been pre-authorized by a payer.

In one embodiment, the identified discovery data may include data that may be similar to the first healthcare claim 500. In some embodiments, the discovery data may include a second healthcare claim 500. The second healthcare claim 500 may include a healthcare claim 500 that includes at least some claim data elements 502 whose values 506 are similar to some of the values 506 of the first healthcare claim 500. The second healthcare claim 500 having similar claim data elements 502 to the first healthcare claim 500 may include the first healthcare claim 500 and the second healthcare claim 500 including similar patient names, patient addresses, patient Social Security numbers, employer names, employer addresses, payer names, payer addresses, or other data. In some embodiments, claim data elements 502 being similar may include the claim data element values 506 matching, the claim data element values 506 being similar (e.g., “John Albert Smith” and “John A. Smith”) within a pre-determined threshold, or may be similar in another way.

For example, the second healthcare claim 500 may include a healthcare claim 500 that includes the same patient as the first healthcare claim 500. The automatic discovery module 208 may determine that the first healthcare claim 500 and the second healthcare claim 500 include the same patient in a variety of ways. In one embodiment, the automatic discovery module 208 may determine that the first healthcare claim 500 and the second healthcare claim 500 include the same patient by determining that one or more claim data elements 502 of the first healthcare claim 500 are similar to one or more claim data elements 502 of the second healthcare claim 500.

In another example of the second healthcare claim 500 including a healthcare claim 500 that includes at least some claim data elements 502 whose values 506 are similar to some of the values 506 of the first healthcare claim 500, the second healthcare claim 500 may include a healthcare claim 500 that includes the same employer as the first healthcare claim 500. Often, patients with the same employer have the same workers' compensation insurance provider or other healthcare insurance provider. Thus, healthcare claims 500 with the same employer but different payer information may indicate that payer-related claim data elements 502 of one of the healthcare claims 500 should be replaced with the payer-related claim elements 502 of the other healthcare claim 500.

In some embodiments, the discovery data may include the data gathered by the data gathering module 210. Such data, as discussed above, may include data retrieved from an external computing device 102(2). Such discovery data may be similar to the first healthcare claim 500 in response to one or more claim data elements 502 being related to the content of the discovery data. As an example, the first healthcare claim 500 may include the value 506 “Academy Sports Plus” for the employer name claim data element 502(2). The first healthcare claim 500 may also not include payer name and address claim data elements 502(4)-(5), or the payer name and address claim data elements 502(4)-(5) may include incomplete data, such as “unknown” or “workers' comp.” The automatic discovery module 208 may (1) determine, from the claim data element 502(2), that Academy Sports Plus is the relevant employer for the first healthcare claim 500, (2) look up the payer information associated with Academy Sports Plus in the data storage 106, as indicated by the data retrieved by the data gathering module 210 from the external computing device 102(2), and (3) select the associated payer information as the identified discovery data. A similar process can also be used for employer information and other claim data elements 502.

The method 400 may include identifying 402 a replacement value 506 for the claim data element value 506 of the first claim data element 502. In some embodiments, identifying 402 the replacement value 506 may include identifying multiple possible replacement values 506. Identifying 402 the replacement value 506 may include selecting identified discovery data, i.e., discovery data identified as described above. Such discovery data may include a claim data element value 506 of a second claim data element 502 from a second healthcare claim 500 or data gathered by the data gathering module 210.

In one embodiment, the discovery data may include a payer-related claim data element 502 such as the payer ID number claim data element 502(3), the payer name claim data element 502(4), or the payer address claim data element 502(5). In some embodiments, the discovery data may include a patient-related claim data element 502 such as the patient name claim data element 502(1), a patient Social Security number claim data element 502, or an employer name claim data element 502(2). The discovery data may include a claim number, injury date, billing location ID, or subscriber relation code claim data element 502.

In some embodiments, the automatic discovery module 208 may select replacement value 506 based on the discovery data differing from the first claim data element's 502 value 506 and other claim data elements 502 being substantially similar. For example, where the discovery data includes a second healthcare claim 500, the first healthcare claim 500 and the second healthcare claim 500 may include the same employer name claim data elements 502(2), but the payer name and payer address claim data elements 502(4) and 502(5) may differ.

The method 400 may include assigning 404 an accuracy score to the replacement value 506. The accuracy score of the replacement value 506 may include a measure of confidence of the automatic discovery module 208 that the replacement value 506 is the correct value 506 for the corresponding claim data element field 504 of the first claim data element 502. The accuracy score may include a percentage (e.g., where 0% indicates no confidence that the replacement value 506 is correct and 100% indicates complete confidence that the replacement value 506 is correct), a decimal number between 0 and 1 (e.g., where 0.0 indicates no confidence that the replacement value 506 is correct and 1.0 indicates complete confidence that the replacement value 506 is correct), or some other value.

In one embodiment, the accuracy score may be based on the similarity of the replacement value 506 to a value 506 of a second claim data element 502 that belongs to a previous, similar healthcare claim 500. For example, if (1) the first claim data element 502 is a “Payer Name” claim data element 502(4) with the value 506 of “Academy Sports Plus,” (2) the replacement value 506 is “Accident Fund, Inc.,” and (3) a similar healthcare claim 500 for the same patient but with the “Payer Name” claim data element 502(4)'s value 506 being “Accident Fund, Inc.” was recently successfully processed, then the automatic discovery module 208 may calculate a relatively high accuracy score for the replacement value 506 of “Accident Fund, Inc.”

In one embodiment, the accuracy score may be based on whether the Social Security number claim data element 502 matches the Social Security number claim data element 502 of a previously successfully processed claim 500 that was successfully paid within a certain time elapsed amount. The time elapsed amount may include 90 days, 180 days, 365 days, or some other amount of time. The time elapsed amount may include time elapsed from the current day, the service date 502(9) of the claim 500 being currently processed, or some other time or date. In certain embodiments, the accuracy score may be based on whether the patient name claim data element 502(1) matches the patient name claim data element 502(1) and the patient birthdate claim data element 502 matches the patient birthdate claim data element 502 of a previously successfully processed claim 500 that was successfully paid within a certain time elapsed amount. In some embodiments, the previous claim 500 may include, instead of a successfully processed claim 500, a claim 500 that was denied for a reason other than eligibility failure.

In some embodiments, assigning an accuracy score to the replacement value 506 may include assigning an accuracy score for each replacement value 506 for the first claim data element 502. Each accuracy score may be different. In some embodiments, assigning an accuracy score may include assigning an accuracy score for the entire claim 500. The entire claim accuracy score may include a mean or median of the accuracy scores of the claim data elements 502 of the claim 500. The entire claim accuracy score may include a weighted average of the accuracy scores of the claim data elements 502 of the claim 500. The entire claim accuracy score may include some other accuracy metric.

In one embodiment, the method 400 may include, in response to the accuracy score exceeding a pre-determined score threshold, replacing 406 the claim data element value 506 of the first claim data element 502 with the replacement value 506. The automatic discovery module 208 may replace 406 the claim data element value 506 automatically and without receiving manual input to confirm the replacement. Replacing 406 the claim data element value 506 may include overwriting the value 506 of the first claim data element 502 after the parsing 304 but before storing the claim data element 502 in the data storage 106. Replacing 406 the claim data element value 506 may include overwriting the value 506 of the first claim data element 502 in the data storage 106.

In some embodiments, the pre-determined score threshold may be based on the claim data element 502. Different claim data elements 502 may include different score thresholds. For example, the pre-determined threshold for the “Patient Name” claim data element 502(1) may be 50% and the pre-determined threshold for “Payer Name” may be 75%. The pre-determined threshold may include 10%, 20%, 30%, 40%, 50%, 60%, 70%, 80%, or some other percentage, numerical value, or the threshold value.

In some embodiments, the pre-determined threshold score may be based on the type of claim 500 associated with the claim data elements 502. For example, a workers' compensation claim 500 may use a pre-determined threshold score of 70%, and a VA claim 500 may use a pre-determined threshold of 60%

In one or more embodiments, the method 400 may include, in response to the accuracy probability score being below the pre-determined score threshold, presenting 408 the replacement value 506 via a user interface. The user interface may include a user interface of a software application in data communication with the server 104. The user interface may be displayed on a computing device in data communication with the server 104.

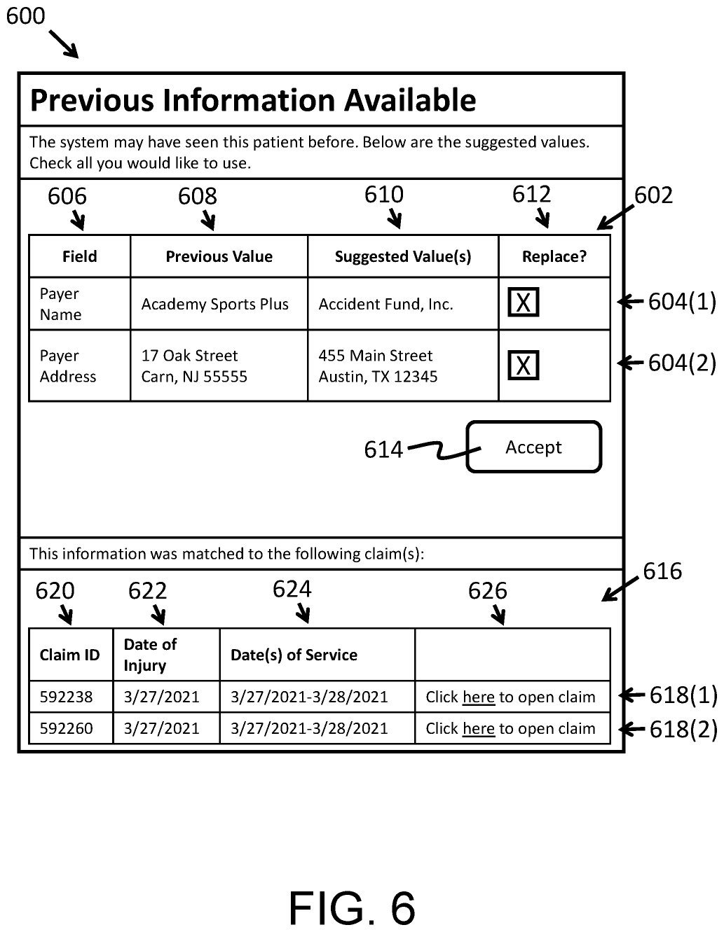

FIG. 6 depicts one example embodiment of a user interface 600. The user interface 600 may include the user interface of the step 408. In one embodiment, the user interface 600 may include a replacement values table 602. The replacement values table 602 may organize a way to present one or more replacement values 506 to a user. The replacement values table 602 may include one or more rows 604(1)-(n).

In some embodiments, a row 604 of the replacement values table 602 may include a claim data element field 606. The claim data element field 606 may indicate the field 504 to which the replacement value 506 pertains. The row 604 may include a previous value 608. The previous value 608 may include the claim data element value 506 that was originally included with the first claim data element 502. The row 604 may include one or more suggested values 610. A suggested value 610 may include a replacement value 506. The replacement value 506 may include a replacement value 506 identified in step 402 of the method 400. The row 604 may include a checkbox 612. In response to the user checking the checkbox 612 and pressing the accept button 614, the automatic discovery module 208 may receive an acceptance from the user interface 600 regarding the replacement value 506 and may replace the claim data element value 506 of the first claim data element 502 with the replacement value 506.

As an example, in FIG. 6, the replacement values table 602 may include two rows: 604(1) and 604(2). The first row 604(1) may pertain to the “Payer Name” field 606. As indicated by the previous value 608 of the first row 604(1), the “Payer Name” claim data element 502(4), as originally generated by the file ingestion module 202, data validation module 204, or data storage module 206 based on the healthcare claim 500, may have included the value 506 “Academy Sports Plus.” However, during the automatic discovery process of the method 400, the automatic discovery module 208 may have identified 402 the value 506 “Accident Fund, Inc.” as a replacement value 506. Thus, the replacement values table 602 may include this replacement value 506 in the suggested value 610 of the second row 604(1). Similarly, the automatic discovery module 208 may have identified 402 the value 506 “455 Main Street, Austin, Tex. 12345” as a replacement value 506 for the “Payer Address” claim data element 502(5) and included the replacement value 506 in the suggested value 610 of the second row 604(2). The user may decide to the accept the replacement values 506 of the first row 604(1) by checking the checkbox 612 of the first row 604(1). The user may also accept the replacement value 506 of the second row 604(2) by checking the corresponding checkbox 612. After the user has decided which checkboxes 612 to check, the user may press the accept button 614 to finalize the decision. In response, the replacement value 506 “Accident Fund, Inc.” may be saved as the value 506 for the claim data element 502(4), and the replacement value “455 Main Street, Austin, Tex. 12345” may be saved as the value 506 for the claim data element 502(5).

The user interface 600 may include a previous information table 616. The previous information table 616 may organize a way to present further information about the suggested values 610 to the user. The further information may include a descriptive explanation indicating why the automatic discovery module 208 identified the replacement value(s) 506 of the replacement values table 602. The further information may include data from a healthcare claim 500 already stored in the data storage 106, claim data elements 502 already stored in the data storage 106, data from an external computing device 102 (e.g., a third-party database), or data from some other data source. The previous information table 616 may include one or more rows 618(1)-(2).

In one embodiment, a row 618 of the previous information table 616 may include one or more claim data element values 506. For example, as depicted in FIG. 6, each of the first row 618(1) and the second row 618(2) may include values 506 corresponding to claim data element field 504 “Claim ID” 620, “Date of Injury” 622, and “Date(s) of Service” 624. The automatic discovery module 208 may have retrieved these values 506 from claim data elements 502 stored in the data storage 106, from an external computing device 102, or from some other source. A row 618 may include a link 626 to the data source of the values 620-624. The link 626 may open a user interface that may display further information from which the values 620-624 were derived. For example, as depicted in FIG. 6, the link 626 may include a link to a previous healthcare claim 500. In response to clicking the link 626, the user may be able to view the healthcare claim 500 or a portion of the claim data elements 502 pertaining to the healthcare claim. The healthcare claim 500 may include the second healthcare claim that was matched to the first healthcare claim 500 during the automatic discovery process of the method 400.

In some embodiments, one or more pieces of information may be absent from the healthcare claim 500. This may result from, for example, the file that included the healthcare claim 500 may have been corrupted during its transmission over the data network 108 or a medical billing personnel forgetting to include certain pieces of information in the healthcare claim 500. This may result in one or more claim data elements 502 being absent from the one or more claim data elements 502(1)-(n) generated based on the healthcare claim 500. In response, the automatic discovery module 208 may identify a value 506 for the absent one or more claim data elements 502. Identifying a value 506 for an absent claim data element 502 may be similar to identifying 402 a replacement value 506 as discussed above in relation to step 402 of the method 400. The automatic discovery module 208 may generate a claim data element 502, and the claim data element 502 may include the identified value 506. The automatic discovery module 208 may include the generated claim data element 502 in the one or more claim data elements 502(1)-(n) generated based on the healthcare claim.

In one embodiment, the server 104 may include an eligibility verification module 212. The eligibility verification module 212 may include a patient eligibility verification API. The patient eligibility verification API may allow an external computing device 102(3) to verify patient or workers' compensation insurance information using the information of the server 104 or the data storage 106. In some embodiments, the API may receive certain patient information or workers' compensation insurance information from an external computing device 102(3) and, in response, the API may send data back to the external computing device 102(3) indicating whether the received patient or workers' compensation insurance information is correct. In certain embodiments, the API may receive certain patient information or workers' compensation insurance information from an external computing device 102(3) and, in response, the API may send further patient or workers' compensation insurance back to the external computing device 102.

As an example, an external computing device 102(3) may send the API a patient's name and Social Security number and the payer name and payer address. The automatic discovery module 208 or the eligibility verification module 212 may calculate an accuracy score for each of these pieces of information received from the external computing device 102(3). In response to the accuracy score for a certain piece of information being above a pre-determined threshold, the API may indicate to the external computing device 102(3) that the piece of information is correct. In response to the accuracy score being below the pre-determined threshold, the API may indicate to the external computing device 102(3) that the piece of information may be incorrect.

In another example, an external computing device 102(3) may send the API a patient's name and Social Security number. The eligibility verification module 212 may determine, from the data elements 502 stored in the data storage 106 and from other data sources, a payer name and a payer address associated with the patient. The API may then send the determined payer name and payer address to the external computing device 102(3).

In some embodiments, one or more modules of the server 104 may include one or more machine learning models that may perform at least a portion of the respective module's functionality. For example, in some embodiments, the automatic discovery module 208 may use a machine learning model to discover relationships between two or more claim data elements 502. One relationship may include the relationship between a certain employer name 502(2) and a payer name 502(4). The machine learning model may learn to associate a certain employer name 502(2) with one or more certain payer names 502(4). In some embodiments, the machine learning model may undergo machine learning training, and the training data may include a list of known employer-to-payer relationships. The list may be derived from a government database or from previously successfully processed claims 500 from the data storage 106.

In some embodiments, after the server 104 performs an autodiscovery process (e.g., the autodiscovery process of step 306 of the method 300 of FIG. 3 or the autodiscovery process of the method 400 of FIG. 4) on a claim 500, the server 104 may send the claim 500 to a validation queue. A user of the server 104 may view at least some of the claim data elements 502 of the claim 500 and may determine whether to validate the claim 500. In response to validating the claim 500, the server 104 may generate and format a claim file that may include at least a portion of the claim data elements 502 of the claim 500 to be sent to a payer for processing. In some embodiments, in response to the accuracy score being above a pre-determined accuracy score threshold (which may include a different threshold than the threshold discussed in relation to FIG. 4), the server 104 may automatically generate and send the claim file to the payer without having a user view the claim 500.

The systems and methods of the disclosure improve the technical field of healthcare claims processing. Specifically, by matching previous claim data elements 502 with claim data elements 502 that may have been entered incorrectly, the server 104 may automatically correct errors in complex workers' compensation claims or VA healthcare claims. The specific matching algorithms discussed herein result in the claims being more accurate, and thus, being processed faster than in prior art systems. Furthermore, the specific algorithms discussed herein that calculate accuracy scores allow the server 104 to automatically augment the claim data elements 502, and, if prudent, allow a human to double check to server's 104 determination. This further results in claims being more accurate. The systems and method disclosed herein also are able to automatically detect errors in the healthcare claims and correct these errors. These improvements allow the methods and systems disclosed herein to process healthcare claims at such a volume that the human mind would not be equipped to process such a volume.

While the making and using of various embodiments of the present disclosure are discussed in detail herein, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatuses, systems, and methods described herein. Such equivalents are considered to be within the scope of this disclosure and may be covered by the claims.

Furthermore, the described features, structures, or characteristics of the disclosure may be combined in any suitable manner in one or more embodiments. In the description contained herein, numerous specific details are provided, such as examples of programming, software, user selections, hardware, hardware circuits, hardware chips, or the like, to provide understanding of embodiments of the disclosure. One skilled in the relevant art will recognize, however, that the disclosure may be practiced without one or more of the specific details, or with other methods, components, materials, apparatuses, devices, systems, and so forth. In other instances, well-known structures, materials, or operations may not be shown or described in detail to avoid obscuring aspects of the disclosure.

These features and advantages of the embodiments will become more fully apparent from the description and appended claims, or may be learned by the practice of embodiments as set forth herein. As will be appreciated by one skilled in the art, aspects of the present disclosure may be embodied as an apparatus, system, method, computer program product, or the like. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present disclosure may take the form of a computer program product embodied in one or more computer-readable media having program code embodied thereon.

In some embodiments, a module may be implemented as a hardware circuit comprising custom (very large-scale integration) VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer-readable media.

In some embodiments, a module may include a smart contract hosted on a blockchain. The functionality of the smart contract may be executed by a node (or peer) of the blockchain network. One or more inputs to the smart contract may be read or detected from one or more transactions stored on or referenced by the blockchain. The smart contract may output data based on the execution of the smart contract as one or more transactions to the blockchain. A smart contract may implement one or more methods or algorithms described herein.

The computer program product may include a computer-readable storage medium (or media) having computer-readable program instructions thereon for causing a processor to carry out aspects of the present disclosure. The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium may include a portable computer diskette, a random access memory (“RAM”), a read-only memory (“ROM”), an erasable programmable read-only memory (“EPROM” or Flash memory), a static random access memory (“SRAM”), a hard disk drive (“HDD”), a solid state drive, a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations or block diagrams of methods, apparatuses, systems, algorithms, or computer program products according to embodiments of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flow chart diagrams included herein are generally set forth as logical flow chart diagrams. As such, the depicted order and labeled steps are indicative of one embodiment of the presented method. Other steps and methods may be conceived that may be equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The schematic flowchart diagrams and/or schematic block diagrams in the Figures illustrate the architecture, functionality, and operation of possible implementations of apparatuses, systems, methods and computer program products according to various embodiments of the present disclosure. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

Thus, although there have been described particular embodiments of the present disclosure of a new and useful systems and methods for predicting missing data values, it is not intended that such references be construed as limitations upon the scope of this disclosure.

What is claimed is:

1. A non-transitory, computer-readable storage medium including executable instructions stored thereon, wherein the executable instructions, when executed by a processor, cause the processor to:
receive a file that includes a first healthcare claim;
parse the first healthcare claim into one or more claim data elements, wherein each claim data element includes
a claim data element field, and
a claim data element value corresponding to the claim data element field; and
perform an automatic discovery process on a first claim data element of the one or more data elements, wherein the automatic discovery process includes
identifying a replacement value for the claim data element value of the first claim data element,
assigning an accuracy score to the replacement value,
in response to the accuracy score exceeding a pre-determined score threshold, replacing the claim data element value of the first claim data element with the replacement value, and
in response to the accuracy probability score being below the pre-determined score threshold, presenting the replacement value via a user interface.

2. The computer-readable storage medium of claim 1, wherein the healthcare claim includes at least one of:
a workers' compensation claim; or
a Veteran's Health Administration claim.

3. The computer-readable storage medium of claim 1, wherein the claim data element field of the first claim data element includes at least one of:
a payer identifier;
a claim number;
a patient first name;
a patient last name;
an employer name;
a patient government ID number;
a date of injury;
a billing location identifier; or
a subscriber relation code.

4. The computer-readable storage medium of claim 1, wherein the processor identifying the replacement value includes the processor identifying the replacement value based on one or more claim data elements stored in a data lake.

5. The computer-readable storage medium of claim 1, wherein the processor identifying the replacement value includes the processor identifying the replacement value based on a robot process automation (RPA) process.

6. The computer-readable storage medium of claim 5, wherein the RPA process includes the processor:
sending virtualized keystrokes to a login screen of a website;
sending virtualized keystrokes to a search screen of the website;
reading the replacement value from a search result screen of the website; and
storing the replacement value in a data storage in data communication with the processor.

7. The computer-readable storage medium of claim 1, wherein the processor performing the automatic discovery process comprises the processor identifying a second healthcare claim, wherein the second healthcare claim includes a successfully processed healthcare claim.

8. The computer-readable storage medium of claim 7, wherein the processor assigning the accuracy score to the replacement value comprises the processor generating an accuracy score based on:
whether the replacement value matches a claim data element of the second healthcare claim; and
whether the second healthcare claim includes a payment date within a pre-determined time threshold.

9. The computer-readable storage medium of claim 8, wherein the claim data element of the second healthcare claim comprises at least one of:
a Social Security number;
a patient name; or
a patient birthdate.

10. A method, comprising:
receiving a file that includes a first healthcare claim;
parsing the first healthcare claim into one or more claim data elements, wherein each claim data element includes
a claim data element field, and
a claim data element value corresponding to the claim data element field; and
performing an automatic discovery process on a first claim data element of the one or more data elements, wherein the automatic discovery process includes
identifying a replacement value for the claim data element value of the first claim data element,
assigning an accuracy score to the replacement value,
in response to the accuracy score exceeding a pre-determined score threshold, replacing the claim data element value of the first claim data element with the replacement value, and
in response to the accuracy probability score being below the pre-determined score threshold, presenting the replacement value via a user interface.

11. The method of claim 10, further comprising, in response to parsing the first healthcare claim, storing the one or more claim data elements in a data storage.

12. The method of claim 10, further comprising:
identifying a claim data element of the one or more claim data elements, wherein the identified claim data element includes an error; and
modifying the identified claim data element to correct the error.

13. The method of claim 10, wherein performing the automatic discovery process comprises identifying a second healthcare claim, wherein the second healthcare claim is similar to the first healthcare claim.

14. The method of claim 13, wherein:
the second healthcare claim includes a second healthcare claim element; and
identifying the replacement value comprises selecting a claim data element value of the second claim data element as the replacement value.

15. The method of claim 10, wherein:
identifying the replacement value includes identifying a plurality of replacement values; and
presenting the replacement value includes presenting the plurality of replacement values via the user interface.

16. The method of claim 10, further comprising:
determining that a second claim data element is absent from the one or more claim data elements;
identifying a value for the second claim data element;
generating the second claim data element, wherein the second claim data element includes the identified value; and
including the second claim data element in the one or more claim data elements.

17. The method of claim 10, wherein presenting the replacement value via the user interface comprises presenting further text information via the user interface, wherein the further text information is based on the replacement value.

18. The method of claim 10, further comprising:
receiving an acceptance from the user interface; and
replacing the claim data element value of the first claim data element with the replacement value.

19. A system, comprising:
a data storage; and
a server in data communication with the data storage, wherein the server includes a computer-readable storage medium including executable instructions stored thereon, wherein the executable instructions of the computer-readable medium, when executed by a processor of the server, cause the processor to:
receive a file that includes a first healthcare claim,
parse the first healthcare claim into one or more claim data elements, wherein each claim data element includes
a claim data element field, and
a claim data element value corresponding to the claim data element field,
store the first healthcare claim in the data storage, and
perform an automatic discovery process on a first claim data element of the one or more data elements, wherein the automatic discovery process includes
identifying a replacement value for the claim data element value of the first claim data element,
assigning an accuracy score to the replacement value,
in response to the accuracy score exceeding a pre-determined score threshold, replacing the claim data element value of the first claim data element with the replacement value, and
in response to the accuracy probability score being below the pre-determined score threshold, presenting the replacement value via a user interface.

20. The system of claim 19, wherein:
the processor of the server performing the automatic discovery process includes the processor identifying a second healthcare claim, wherein the second healthcare claim is similar to the first healthcare claim;
the second healthcare claim includes a second healthcare claim element; and
identifying the replacement value comprises selecting a claim data element value of the second claim data element as the replacement value.
